(19) United States
(12) Patent Application Publication
(10) Pub. No.: US 2016/0367755 A1
(43) Pub. Date: Dec. 22, 2016

(54) SYSTEM AND METHOD FOR NEUROENDOCRINE CONTROL

(71) Applicant: Massachusetts Institute of Technology, Cambridge, MA (US)

(72) Inventors: Rose T. Faghih, Cambridge, MA (US); Emery N. Brown, Brookline, MA (US); Munther A. Dahleh, Cambridge, MA (US)

(73) Assignee: Massachusetts Institute of Technology, Cambridge, MA (US)

(21) Appl. No.: 15/170,900

(22) Filed: Jun. 1, 2016

Related U.S. Application Data
(60) Provisional application No. 62/168,959, filed on Jun. 1, 2015.

(51) Int. Cl. A61M 5/172; A61K 31/573

(52) U.S. Cl. CPC A61M 5/1723; A61K 31/573; A61M 2005/1726

(57) ABSTRACT

The present invention relates to systems and methods for detecting and regulating neuroendocrine control. The systems involve measurement of real time pulsatile endocrine hormone levels in a subject and calculation of an appropriate dose of an analyte for treating the subject based on several factors. The systems are preferably closed loop systems.

### RELATED APPLICATIONS

This Application claims priority under 35 U.S.C. §119(e) to U.S. Provisional Application Ser. No. 62/168,959, entitled “SYSTEM AND METHOD FOR NEUROENDOCRINE CONTROL” filed on Jun. 1, 2015, which is herein incorporated by reference in its entirety.

### FEDERALLY SPONSORED RESEARCH

This invention was made with government support under Grant No. EFRI-0735956 awarded by the National Science Foundation and government support under Grant No. NIH-0836720 awarded by the National Institutes of Health. The government has certain rights in the invention.

### BACKGROUND OF THE INVENTION

The principle oscillator of circadian rhythms in humans is in the suprachiasmatic nucleus. This master oscillator is responsible for the sleep-wake cycle and hormonal rhythms (e.g. cortisol and melatonin). Disturbance of the central clock may occur as a result of either an environmental change (an individual moves to a different time zone, does shift work, or change in season) or as a result of disease either directly effecting the central oscillator or altering the circadian rhythm or hormones such as cortisol. The loss of circadian rhythms results in diseases for example depression and inflammatory disorders. Biological clocks in tissues that are regulated by glucocorticoids, for example cortisol, could include the brain, endocrine, immune system, lungs, cardiovascular system, genitor-urinary system, reproductive system.

### SUMMARY OF THE INVENTION

The present invention relates to methods for delivering cortisol to a subject having adrenal insufficiency comprising detecting a real-time level of circulating cortisol in blood of a subject having adrenal insufficiency, administering intermittent doses of cortisol to the subject based the level of circulating cortisol wherein the cortisol is within a lower circadian limit of cortisol and wherein the dose of cortisol administered to the subject adjusts the dose of cortisol to a range within an upper circadian limit of cortisol. In some embodiments the subject has Addison's disease.

In some embodiments the intermittent doses are pulses. In other embodiments the dose of cortisol is at a maximum of the upper circadian limit of cortisol. In other embodiments the upper and lower circadian limits of cortisol are based on upper and lower circadian limits of cortisol in healthy humans.

A close loop cortisol infusion system is provided according to other aspects of the invention. The system includes a sensor that measures cortisol levels in real time in a subject having adrenal insufficiency, a control algorithm that determines the amount of cortisol needed to keep cortisol levels within a healthy range, and a cortisol infusion device for delivering intermittent doses of cortisol to the patient in response to the calculation of the required cortisol.

In some embodiments the intermittent doses of cortisol are within a range between a lower circadian limit of cortisol and an upper circadian limit of cortisol. In other embodiments the intermittent doses of cortisol are at a maximum upper circadian limit of cortisol.

The amount of cortisol needed to keep cortisol levels within a healthy range is calculated in some embodiments based on an impulsive system. In other embodiments the amount of cortisol needed to keep cortisol levels within a healthy range is calculated based on a switched system. The switched system may calculate different infusion rates. Alternatively or additionally the switched system may calculate different clearance rates.

In some embodiments the amount of cortisol needed to keep cortisol levels within a healthy range is calculated based on a cortisol input amount. In other embodiments the cortisol input amount is calculated based on an amount of cortisol that is naturally produced by the body and an amount of cortisol that is delivered from the close loop cortisol infusion system.

A system for the delivery of an analyte to a subject is provided in other aspects of the invention. The system comprises a pulsatile endocrine hormone sensor configured to provide a sensor pulsatile endocrine hormone measurement signal representative of sensed pulsatile endocrine hormone; an analyte delivery device configured to deliver intermittent doses of an analyte to a subject in response to control signals; and a controller programmed to receive the sensor pulsatile endocrine hormone measurement signal and to provide a delivery control signal to the delivery device as a function of the received sensor pulsatile endocrine hormone measurement signal in accordance with a control model.

In some embodiments the control model is a range of circadian levels of pulsatile endocrine hormone in a healthy human. In other embodiments the range of circadian levels of pulsatile endocrine hormone has a lower limit and an upper limit and wherein the delivery control signal delivers a signal to provide a dose near the upper limit of the range. In yet other embodiments the analyte is cortisol, a cortisol antagonist, a cortisol agonist, growth hormone, a growth hormone agonist, or a growth hormone antagonist. In some embodiments the pulsatile endocrine hormone is cortisol. In yet other embodiments the pulsatile endocrine hormone is growth hormone.

The pulsatile endocrine hormone in other embodiments is progesterone, follicle stimulating hormone (FSH), Luteinizing hormone (LH) or thyroid hormone. In some embodiments the control model is a range of cyclical but infradian levels of pulsatile endocrine hormone in a healthy human. In other embodiments the range of infradian levels of pulsatile endocrine hormone has a lower limit and an upper limit and wherein the delivery control signal delivers a signal to provide a dose near the upper limit of the range. In some embodiments the delivery control signal is also a function of subject specific properties including health or weight of the subject and a basal pulsatile endocrine hormone profile.

The controller, in some embodiments, is also programmed to calculate from the control model an accepted value; the controller is programmed to calculate from the pulsatile endocrine hormone level signal an inferred value; the controller is programmed to forecast a future pulsatile endocrine hormone level excursion based on the accepted value and inferred value; and the controller is also programmed to adjust the delivery control signal in accordance with the forecast future plasma pulsatile endocrine hormone level excursion. In some embodiments the inferred value comprises pulsatile endocrine hormone flux. In other embodiments the controller is also programmed to adjust a value of the delivery control signal in accordance with a safety check.

A device comprising a pulsatile endocrine hormone sensor configured to provide a sensor pulsatile endocrine hormone measurement signal representative of sensed pulsatile endocrine hormone is provided in other aspects of the invention. In some embodiments the sensor further comprises a transmitter unit coupled to the sensor.

According to other aspects the invention is a device comprising a controller programmed to receive a sensor pulsatile endocrine hormone measurement signal from a sensor and to provide a delivery control signal to a delivery device as a function of the received sensor pulsatile endocrine hormone measurement signal. In some embodiments the controller further comprises a receiver unit coupled to the controller.

Each of the embodiments of the invention can encompass various recitations made herein. It is, therefore, anticipated that each of the recitations of the invention involving any one element or combinations of elements can, optionally, be included in each aspect of the invention.

### BRIEF DESCRIPTION OF THE DRAWINGS

FIGS. 1A-1B show cortisol levels and control obtained using Example 1. FIG. 1A displays the optimal cortisol profile (black curve), constant upper bound, and constant lower bound. FIG. 1B displays the optimal control. The optimization problem obtained 12 impulses over 24 h as the optimal control (the timing of the control was discretized into 1440 points; the obtained control takes 12 non-zero values, i.e., impulses, while it is zero everywhere else). The optimization problem was solved using the parameters given in Example 1 in Table 1 and the upper and lower bounds provided in Tables 2 and 3, respectively.

FIGS. 2A-2C show cortisol levels and control obtained using Example 2. FIG. 2A displays the optimal cortisol profile obtained by adding a zero mean Gaussian measurement error with a standard deviation of σ=0.45 to each simulated data point; the cortisol levels are recorded every 10 min. FIG. 2B displays the optimal cortisol profile (black curve), two-harmonic upper bound, and two-harmonic lower bound; the cortisol levels are recorded every minute. FIG. 2C displays the optimal control. The optimization problem obtained 16 impulses over 24 h as the optimal control (the timing of the control was discretized into 1440 points; the obtained control takes 16 non-zero values, i.e., impulses, while it is zero everywhere else). The optimization problem was solved using the parameters given in Example 2 in Table 1 and the upper and lower bounds provided in Tables 2 and 3, respectively.

FIGS. 3A-3C show cortisol levels and control obtained using Example 3. FIG. 3A displays the cortisol profile obtained by adding a zero mean Gaussian measurement error with a standard deviation of σ=0.45 to each simulated data point; the cortisol levels are recorded every 10 min. FIG. 3B displays the obtained cortisol profile (black curve), two-harmonic upper bound, and two-harmonic lower bound. FIG. 3C displays the obtained control. The optimization problem obtained 16 impulses over 24 h as the control (the timing of the control was discretized into 1440 points; the obtained control takes 16 non-zero values, i.e., impulses, while it is zero everywhere else). The optimization problem was solved using the parameters given in Example 3 in Table 1 and the upper and lower bounds provided in Tables 2 and 3, respectively.

FIGS. 4A-4C cortisol levels and control obtained using Example 4. FIG. 4A displays the cortisol profile obtained by adding a zero mean Gaussian measurement error with a standard deviation of σ=0.45 to each simulated data point; the cortisol levels are recorded every 10 min. FIG. 4B displays the obtained cortisol profile (black curve), two-harmonic upper bound, and two-harmonic lower bound. FIG. 4C displays the obtained control. The optimization problem obtained 12 impulses over 24 h as the control (the timing of the control was discretized into 1440 points; the obtained control takes 12 non-zero values, i.e., impulses, while it is zero everywhere else). The optimization problem was solved using the parameters given in Example 4 in Table 1 and the upper and lower bounds provided in Tables 2 and 3, respectively.

FIG. 5 is a flow chart showing an exemplary decision tree for calculating ideal dose at a time point for delivery of analyte in intermittent fashion.

FIG. 6 is a schematic of an embodiment of a system of the invention.

### DETAILED DESCRIPTION OF THE INVENTION

Currently, hormonal deficiencies are treated by continuous dosing methods. For example, a patient who suffers from Addison's disease (cortisol deficiency) takes cortisone once or twice a day for their cortisol deficiency. The methods are not optimal since cortisol is typically released in a non-continuous manner in healthy subjects. In a healthy subject there are 15 to 22 secretory events that lead to the observed cortisol levels over 24 hours.

Many hormones that have been well-investigated appear to be released in pulses (Stavreva et al., 2009); for example, cortisol, gonadal steroids, and insulin are released in a pulsatile manner (Veldhuis, 2008). Pulsatility is a physiological way of increasing hormone concentrations rapidly and sending distinct signaling information to target cells (Veldhuis, 2008). Ultradian pulsatile hormone secretion allows for encoding information via both amplitude and frequency modulation and is a way of frequency encoding (Lightman and Conway-Campbell, 2010; Walker et al., 2010b). Pulsatile signaling permits target receptor recovery, rapid changes in hormone concentration, and greater control, and is also more efficient than continuous signaling (Walker et al., 2010b). The mechanism underlying the generation of hormone pulses and why this method of signaling is chosen by the body over continuous signaling is not known.

The transcriptional program prompted by hormone pulses is considerably different from constant hormone treatment (Stavreva et al., 2009). Hormone pulsatility underlies multiple physiological processes. For example, (i) cortisol oscillations have crucial effects on target cell gene expression and glucocorticoids receptor function (McMaster et al., 2011; Walker et al., 2012). (ii) Some psychiatric and metabolic diseases are associated with changes in cortisol pulsatility (Walker et al., 2010a). (iii) When the same amount of corticosterone is administered by constant infusion rather than a pulsatile infusion, it results in a noticeably reduced ACTH response to stress (Lightman and Conway-Campbell, 2010).

The methods of the invention uncover the pulsatile release of hormones (i.e. stress hormones such as cortisol and growth hormone as well as other non-stress endocrine hormones) and propose a novel mathematical formulation that characterizes pulsatile hormonal secretion. The system of the invention is useful as a synthetic impulse controller to mimic the physiology of a healthy subject so that a subject having a hormonal deficit can maintain hormonal levels (e.g. cortisol levels) that are similar to healthy subjects. The system has a variety of clinical utilities including, for instance, controlling cortisol levels in subjects having Addison's disease, controlling growth hormone levels in children with growth deficiency such that they will have normal growth, or controlling progesterone levels in women with infertility to enhance their ability to have children.

The system in some aspects of the invention consists of a sensor that measures pulsatile endocrine hormone levels in real-time in a subject, a control algorithm that identifies hormone levels within a healthy range for the subject at that time and an infusion or delivery device for delivering the appropriate dose of an analyte to the subject.

A pulsatile endocrine hormone as used herein, is an endocrine hormone that is naturally released in intermittent schedules or as pulses, rather than continuously. Pulsatile endocrine hormones include but are not limited to cortisol, progesterone, growth factor, LH, FSH, and thyroid hormone. Some pulsatile endocrine hormones are circadian and others are not. For instance cortisol and growth hormone are regulated by the circadian rhythm. progesterone, LH, FSH, and thyroid hormone are not on the circadian rhythm. Some hormones are on a longer cycle.

A circadian rhythm is a biological process that displays an endogenous, entrainable oscillation of approximately 24 hours. Ultradian rhythms are rhythms that have a period shorter than 24 hours. Infradian rhythms are rhythms that are longer than 24 hours. These can be rhythms that exceed 24 hours by a few hours; they may be cycles of a few days, a few weeks, a few months, a year or even of many years.

A subject is a mammal, including humans and non-human mammals. In some embodiments the subject is a human such as a human patient that has a hormonal deficiency. A human patient having a hormonal deficiency is a patient that has an imbalance in hormone levels in comparison to a healthy human subject that has hormonal levels within a normal range.

Thus, the subject may be a patient having a disease or condition that is associated with a hormonal deficiency. Diseases or conditions associated with hormonal deficiency include but are not limited to adrenal deficiencies, such as Adrenal insufficiency and adrenal overproduction. Adrenal insufficiency is a condition in which the adrenal glands do not produce adequate amounts of steroid hormones, primarily cortisol; but may also include impaired production of aldosterone (a mineralocorticoid).Addison's disease and congenital adrenal hyperplasia are forms of adrenal insufficiency. Adrenal insufficiency may also arise when the hypothalamus or the pituitary gland does not make adequate amounts of the hormones that assist in regulating adrenal function. This is called secondary or tertiary adrenal insufficiency and is caused by lack of production of ACTH in the pituitary or lack of CRH in the hypothalamus, respectively.

Adrenal overproduction include diseases associated with excess levels of the hormone cortisol which are responsible for Cushing syndrome. When the level of cortisol is too high in the body, Cushing syndrome may develop. Cushing syndrome can develop from a cause outside of your body (exogenous Cushing syndrome), for example, by taking oral corticosteroid medications in high doses over an extended period of time. These medications, such as prednisone, have the same effect in the body as does cortisol produced by your body. It's also possible to develop Cushing syndrome from injectable corticosteroids, for example, repeated injections for joint pain, bursitis and back pain. Inhaled steroid medicines and steroid skin creams may cause Cushing syndrome, especially if taken in high doses. Cushing syndrome may also be due to the body's own overproduction of cortisol (endogenous Cushing syndrome). This may occur from excess production by one or both adrenal glands, or overproduction of the adrenocorticotropic hormone (ACTH), which normally regulates cortisol production, In these cases, Cushing syndrome may be related to: a pituitary gland tumor (pituitary adenoma), an ectopic ACTH-secreting tumor, primary adrenal gland disease, cancerous tumors of the adrenal cortex (adrenocortical carcinomas, or Familial Cushing syndrome.

Diseases or conditions associated with hormonal deficiency also include but are not limited to diseases associated with imbalance in levels of thyroid hormone, growth factor, progesterone, FSH, or LH. The thyroid gland manufactures hormones that regulate the body's metabolism (the process of creating and using energy). There are several different disorders that can arise when the thyroid produces too much hormone (hyperthyroidism) or not enough (hypothyroidism). Several common thyroid disorders include Hashimoto's disease, Graves' disease, goiter, and thyroid nodules. Hashimoto's disease is also known as chronic lymphatic thyroiditis and is a common cause of hypothyroidism. Graves' disease is a common cause of hyperthyroidism.

Diseases or conditions associated with progesterone, FSH, and LH imbalance include disorders of the reproductive system and bones. For instance, miscarriages, infertility, endometriosis, inflammatory diseases, and osteoporosis. The methods and systems of the invention may be useful for treating any of these diseases. For instance the hormones can be delivered in the appropriate amounts and at the appropriate times based on the calculations of the invention to treat the infertility and avoid miscarriages as well as the other diseases.

Diseases or conditions associated with Growth Hormone (GH) are also treatable according to the methods and systems of the invention. GH is the pituitary hormone that stimulates body growth, increased height and development during childhood. In adulthood, growth hormone plays a role in maintaining normal body composition, including muscle mass, normal bone strength and optimal quality of life. levels are increased during acute physical stress. The level can increase up to two- to tenfold. GH may enhance metabolic activity. In psychological stress, there is GH secretory defect. GH deficiency is most commonly observed in conjunction with other pituitary hormone deficiencies. This usually occurs in patients who have had pituitary tumors, surgery and/or radiation and also occur as a complication of traumatic brain injury.

The disease and conditions described herein may be treated with an analyte. The appropriate amount of analyte useful for treating the disease or condition at a particular time point is calculated using the devices and systems described herein. Analytes are compounds useful for treating the disease or conditions associated with pulsatile endocrine hormone imbalances. Analytes include but are not limited to cortisol, cortisol agonists, cortisol antagonists, thyroid hormone (T4 or T3), thyroid hormone agonists, thyroid hormone antagonists, growth factor, growth factor agonists, growth factor antagonists, progesterone, progesterone agonists, progesterone antagonists, FSH, FSH agonists, FSH antagonists, LH, LH agonists, or LH antagonists

Specific antagonists of cortisol activity are known in the art. WO9917779 describes the use of glucocorticoid receptor antagonists to ameliorate psychotic disorders. Cortisol is a glucocorticoid which binds an intracellular glucocorticoid receptor and elevated levels of cortisol, or hypercortisolemia, can be controlled by blocking the activity of the receptor to which cortisol binds. An example of a cortisol receptor antagonist is mifepristone and WO9917779 teaches the use of this antagonist to treat conditions that result from elevated cortisol levels. A further example is described in WO02076390 which teaches the use of glucocorticoid receptor antagonists to treat stress conditions, for example post traumatic stress disorder, in individuals.

The levels of pulsatile endocrine hormone are detected with a sensor. Sensors for detecting hormone levels are known in the art. The sensor may be an external sensor or an implantable sensor, as long as the sensor is able to determine real time levels of circulating hormone. A real time level of circulating hormone refers to a level of hormone that is present within the blood and is detected within a 30 minute period. In some embodiments the real time level of circulating hormone is detected and used in the methods or systems of the invention within 30, 25, 20, 15, 14, 13, 12, 11, 10, 9, 8, 7, 6, 5, 4, 3, 2, or 1 minute of the actual hormone circulating at that level. In some embodiments the real time measurement is taken within 30 seconds or simultaneously with the actual hormone circulating at that level.

An external sensor may be a sensor that detects hormone level in a blood sample removed from a patient at a time point. The external sensor may communicate with a controller program to calculate the appropriate dosage of the analyte to be delivered at that time point. A signal may then be delivered to an implantable device to instruct the device to release the analyte at the appropriate dosage or the appropriate dosage may be manually administered to the subject at the appropriate dosage.

An implantable sensor may be implanted into a tissue i.e. for instance under a skin surface of a human and in fluid contact with a bodily fluid for a duration of sensor life. The sensor is adapted to sample an hormone level in the bodily fluid; wirelessly transmitting a data signal from a transmitter coupled with the hormone sensor to a receiver device, the data signal corresponding to an hormone level sampled by the hormone sensor; determining, at the receiver device which is optionally a controller programed to receive the sensor signal, the hormone level using the data signal received from the hormone sensor; calculating the appropriate dosage, and instructing a delivery device in the form of a delivery control signal to deliver the analyte.

The sensor may be, for example, subcutaneously positioned in a patient for the continuous or periodic monitoring of an hormone in a patient's interstitial fluid. This may be used to infer the hormone level in the patient's bloodstream. The sensors also may be inserted into a vein, artery, or other portion of the body containing fluid. A sensor of the subject disclosure may be configured for monitoring the level of the hormone over a time period which may range from hours, days, weeks, or longer.

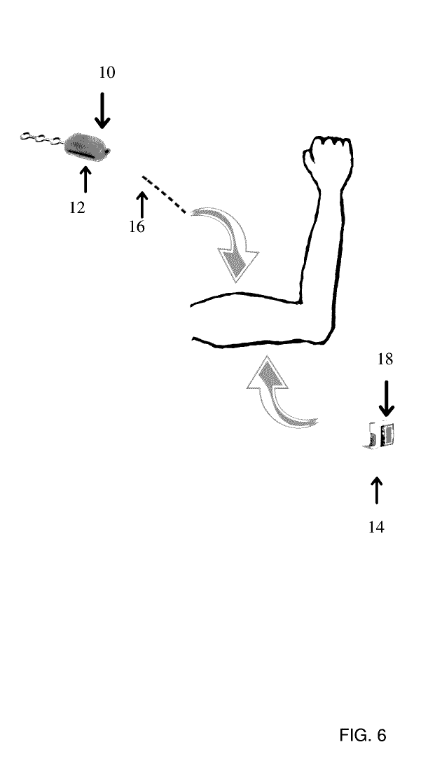

The sensor may be part of a closed loop system (FIG. 6). A close loop system is a system that works autonomously with a sensor (10) detecting levels of circulating hormone and generating a signal to reflect those levels, a controller (12) that receives those signals and processes the calculations described herein to identify the appropriate dosage of analyte at that time, and a delivery device (14) which responds to the controller and delivers the analyte to the subject. The system does not require human intervention to operate.

A transmitter unit (16) may also be coupled to the sensor. A receiver unit (18) may be configured to communicate with the transmitter unit via a communication link. The receiver unit may be further configured to transmit data to a data processing terminal for evaluating the data received by the receiver unit. Moreover, the data processing terminal in one embodiment may be configured to receive data directly from the transmitter unit via a communication link which may optionally be configured for bi-directional communication. Some or all of the various components may be separate components, or some or all may be integrated into a single unit. The system may include one or more sensors, transmitter units, receiver units, communication links, and delivery devices.

The controller programed to receive signal may include a personal computer, a portable computer such as a laptop or a handheld device (e.g., personal digital assistants (PDAs)), and the like, each of which may be configured for data communication with the receiver via a wired or a wireless connection. The controller may also linked to or include an infusion device or delivery device such as an analyte infusion pump or the like, which may be configured to administer analyte to patients, and which may be configured to communicate with the receiver unit for receiving, among others, the measured hormone level signal.

Additionally, the transmitter unit, the controller and the delivery device may each be configured for bi-directional wireless communication such that each component may be configured to communicate (that is, transmit data to and receive data from) with each other via a wireless communication link. In one embodiment, the communication link 103 may include one or more of an RF communication protocol, an infrared communication protocol, a Bluetooth enabled communication protocol, an 802.11x wireless communication protocol, or an equivalent wireless communication protocol which would allow secure, wireless communication of several units (for example, per HIPAA requirements) while avoiding potential data collision and interference.

The sensors are adapted to periodically (or intermittently) monitor hormone levels for a period of time, at time intervals, e.g., usually 15-22 times in a 24 hour period.

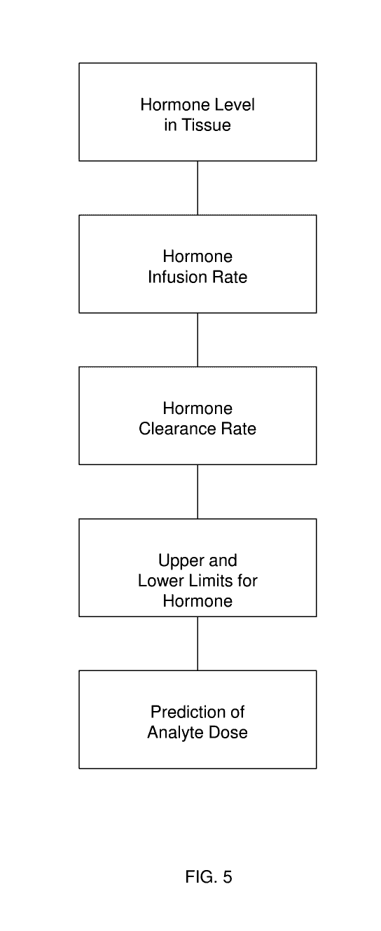

Once the sensor detects hormone levels, the data is analyzed in an algorithm to determine the appropriate dosage of analyte to be administered to the subject at the time period. FIG. 5 is a flow chart depicting an example of the decisions which can be made to determine the dosage. As described above the hormone level may be measured in real time in the tissue of the subject. In addition to the real time hormone levels the upper and lower limits for the hormone at the particular time point in the hormones cycle from a healthy subject are considered. The levels from a healthy subject may be from a single healthy subject or may be a combined value such as an average of levels found in several or numerous healthy subjects. The hormone levels fluctuate with time in a normal healthy subject. For instance a hormone such as cortisol fluctuates with a circadian rhythm. The upper and lower levels of cortisol levels may be established according to the circadian rhythm. This information is incorporated into the calculation. Additionally, the calculation may involve an impulsive system or a switched system. A switched system involves calculating infusion and clearance rates to help understand how the detected hormone level will change with time and how that compares to the upper and lower levels of the healthy subject. These factors are useful for predicting the hormone level and consequently the appropriate analyte dosage to treat the subject.

To calibrate the system, the model parameters for a patient with hormone deficiency were identified. Then, a set of lower and upper bounds for a desired healthy range are set and tailored to the conditions of the individual patient. Then, using a mathematical formulations, such as one of the formulations set forth below or otherwise described herein, the timing and amplitudes of the dose to be injected to the patient to maintain the hormone levels within the healthy range were calculated.

Exemplary Mathematical Formulas of the present disclosure include the following:

### Formulation 1

| | | |
|---|---|---|
| | | min\|\|u\|\| ₀ |
| s.t. | | |
| | u(t) ≧ 0 | |

x(t) = Ax(t) + Bu(t)
h(t) ≦ x(t) ≦ q(t)

### Formulation 2:

### Dynamical System Formulation (Hybrid System)

$$\{ \begin{matrix}x ( t ) = f ( x , t ) , if h ( {τ}_{i}^{-} ) < x ( {τ}_{i}^{-} ) < q ( {τ}_{i}^{-} ) \\ ( Continuous dynamics - no control within bounds ) \\ \\ {x}_{i}^{+} = {x}_{i}^{-} + g ( {x}_{i}^{-} ) {u}_{i} , if x ( {τ}_{i}^{-} ) \leq h ( {τ}_{i}^{-} ) or q ( {τ}_{i}^{-} ) \leq x ( {τ}_{i}^{-} ) \\ ( Jump dynamics - pulse control to bring the system within bounds )\end{matrix}$$

### Minimization Cost Function (Both Continuous and Jump Dynamics)

$$J = \underbrace{{\sum }_{t = 1}^{K + 1} ( {\int }_{{τ}_{i - 1}^{+}}^{{τ}_{i}^{-}} {x ( t )}^{′} Qx ( t ) d t )}_{Energy in State} + \underbrace{{\sum }_{t = 1}^{K} ( {u}_{t}^{′} {Ru}_{i} )}_{Energy in Control}$$

This is a continuous time system in which a pulse causes a jump in the system once the state reaches/passes over the bounds so that the state is kept/brought back within the bounds.

### Formulation 3:

### Dynamical System Formulation (Switched System)

$${x}_{t + 1} = \{ \begin{matrix}{Ax}_{t} , {h}_{t} < {x}_{t} < {q}_{t} & ( no control within bounds ) \\ {Ax}_{t} + {Bu}_{t} , otherwise & ( pulse control to keep the system within bounds\end{matrix}$$

### Cost Function

$$J = \underbrace{{\sum }_{t = 1}^{T} ( {x}_{t}^{′} {Qx}_{t} )}_{Energy in State} + \underbrace{{\sum }_{t = 1}^{K} ( {u}_{t}^{′} {Ru}_{t} )}_{Energy in Control}$$

This is a discrete time system in which a pulse causes a jump in the system once the state reaches/passes over the bounds so that the state is kept/brought back within the bounds

Exemplary embodiments of the invention are described below with respect to cortisol. The description is exemplary only and is not limiting. Each of these embodiments are applicable to the other pulsatile endocrine hormones in addition to cortisol.

Cortisol is released to relay information to cells to regulate metabolism and reaction to stress and inflammation. In particular, cortisol is released in the form of pulsatile signals. This low-energy method of signaling seems to be more efficient than continuous signaling. We hypothesize that there is a controller in the anterior pituitary that leads to pulsatile release of cortisol, and propose a mathematical formulation for such controller, which leads to impulse control as opposed to continuous control. We postulate that this controller is minimizing the number of secretory events that result in cortisol secretion, which is a way of minimizing the energy required for cortisol secretion; this controller maintains the blood cortisol levels within a specific circadian range while complying with the first order dynamics underlying cortisol secretion. We use an l₀-norm cost function for this controller, and solve are weighed l₁-norm minimization algorithm for obtaining the solution to this optimization problem. We use four examples to illustrate the performance of this approach: (i) a sample non-physiological problem that achieves impulse control, (ii) two examples that achieve physiologically plausible pulsatile cortisol release, (iii) an example where the number of pulses is not within the physiologically plausible range for healthy subjects while the cortisol levels are within the desired range. This novel approach results in impulse control where the impulses and the obtained blood cortisol levels have a circadian rhythm and an ultradian rhythm that are in agreement with the known physiology of cortisol secretion.

Cortisol is released from the adrenal glands in pulses in response to pulsatile release of ACTH. CRH induces the release of ACTH. In return, cortisol has a negative feedback effect on ACTH and CRH release at the pituitary and hypothalamic levels. The timing and amplitudes of cortisol pulses vary throughout the day where the amplitude variations are due to the circadian rhythm underlying cortisol release with periods of 12 and 24 h (Faghih et al., 2011), and the variations in the timing of cortisol pulses result from the ultradian rhythm underlying cortisol release. Between 15 and 22 secretory pulses of cortisol are expected over 24 h (Veldhuis et al., 1989; Brown et al., 2001).

It was discovered herein that pulsatile release of CRH from the hypothalamus results in pulsatile release of cortisol. Walker et al. suggested that a sub-hypothalamic pituitary-adrenal system results in the pulsatile ultradian pattern underlying cortisol release (Walker et al., 2012) because inducing constant CRH levels resulted in a pulsatile cortisol profile (Walker et al., 2012) while constant ACTH levels did not result in pulsatile cortisol secretion (Spiga et al., 2011). Spiga et al. suppressed the activity of the HPA axis by oral methylprednisolone and infused both constant amounts and pulses of ACTH to test the hypothesis that pulsatile ACTH release is necessary for pulsatile cortisol secretion (Spiga et al., 2011). While pulsatile ACTH resulted in pulsatile cortisol secretion, constant infusion of same amounts of ACTH did not activate cortisol secretion (Spiga et al., 2011). Moreover, studies on sheep in which the hypothalamus has been disconnected from the pituitary suggest that pulsatile input from hypothalamic secretagogues (e.g., CRH or vasopressin) is not necessary for the ultradian rhythm in cortisol secretion or for pulsatile cortisol secretion and pulsatile cortisol secretion is still maintained (Walker et al., 2010a).

Pulsatile cortisol release is controlled by the dynamics in the anterior pituitary. We describe herein the discovery that there is a controller in the anterior pituitary that controls the pulsatile secretion of cortisol and the ultradian rhythm of the pulses via the negative feedback effect of cortisol on the anterior pituitary. As a result, devices which achieve impulse control are also described herein. In optimal control theory, impulse control is a special case of bang-bang control, in which an action leads to instantaneous changes in the states of the system (Sethi and Thompson, 2000). Impulse control occurs when there is not an upper bound on the control variable and an infinite control is exerted on a state variable in order to cause a finite jump (Sethi and Thompson, 2000). Minimizing an l0-norm cost function can achieve impulse control and we used a reweighed l1-norm formulation as a relaxation to the l0-norm to solve the proposed optimization formulation. Moreover, we considered the first-order dynamics underlying cortisol synthesis and the circadian amplitude constraints on the cortisol levels when formulating the optimization problem.

A physiologically plausible optimization problem for cortisol secretion is presented herein by making the following assumptions: (1) Cortisol levels can be described by first-order kinetics for cortisol synthesis in the adrenal glands, cortisol infusion to the blood, and cortisol clearance by the liver described in Brown et al. (2001), Faghih (2010), and Faghih et al. (2011, 2014). (2) There is a time-varying cortisol demand [h(t)] that should be satisfied throughout the day, which is a function of the circadian rhythm. (3) There is a time-varying upper bound on the cortisol level [q(t)] that is a function of the upper bound on the cortisol level that the body can produce or a holding cost so that the cortisol level would not be much above the demand. (4) Control that results in cortisol secretion [u(t)] is non-negative. (5) The body is minimizing the number of resources (control) throughout the day. Hence, we postulate that there is a controller in the anterior pituitary that controls cortisol secretion via the following optimization formulation:

$$\begin{matrix}{min}_{u} {‖ u ‖}_{0} & ( 1 ) \\ s . t . \\ u ( t ) \geq 0 \\ \frac{d {x}_{1} ( t )}{d t} = - λ {x}_{1} ( t ) + u ( t ) \\ \frac{d {x}_{2} ( t )}{d t} = λ {x}_{1} ( t ) - γ {x}_{2} ( t ) \\ h ( t ) \leq {x}_{2} ( t ) \leq q ( t ) & \end{matrix}$$

where x₁ is the cortisol concentration in the adrenal glands and x₂ is the blood cortisol concentration. λ and γ, respectively, represent the infusion rate of cortisol from the adrenal glands into the blood and the clearance rate of cortisol by the liver.

Considering the known physiology of de novo cortisol synthesis (i.e., no cortisol is stored in the adrenal glands) (Brown et al., 2001), we assume that the initial condition of the cortisol level in the adrenal glands is zero [x₁(0)=0] (Brown et al., 2001). Assuming that the input and the states are constant over 1-min intervals, and y₀ is the initial condition of the blood cortisol concentration, blood cortisol levels at every minute over N min can be represented in discrete form by y=[y₁ y₂ . . . y_{N}]′ where yₖ is the blood cortisol level at time k and y can be represented as:

y=Fy0+Gu (2)

where

$$F = {[ \begin{matrix}{f}_{1} & {f}_{2} & … & {f}_{N}\end{matrix} ]}^{′} , {f}_{k} = {e}^{- γ k} , G = {[ \begin{matrix}{g}_{1} & {g}_{2} & … & {g}_{N}\end{matrix} ]}^{′} , \\ {ℊ}_{k} = {[ \begin{matrix}\frac{λ}{λ - γ} ( {e}^{- γ k} - {e}^{- λ k} ) & … & \frac{λ}{λ - γ} ( {e}^{- γ } - {e}^{λ} ) 0 \underbrace{ … 0}_{N - k}\end{matrix} ]}^{′} ,$$

and u represents the control over N minutes. Then, by letting h=[h₁ h₂ . . . h_{N}]′ where hₖ is the cortisol demand at an integer minute k and q=[q₁ q₂ . . . q_{N}]′ where qₖ is the upper bound at the integer minute k. Hence, we solve the discrete analog of the formulation in Equation (1):

$${min}_{u , {x}_{0}} {‖ u ‖}_{0} \\ s . t . \\ u \geq 0 \\ x = {Fy}_{0} + Gu \\ h \leq x \leq q \qquad ( 3 )$$

l₀ problems are generally NP-hard, and instead an l₁-norm relaxation of such problems can be solved. In solving l₁-norm problems, there is a dependence on the amplitude of the coefficients over which the l₁-norm is minimized, and there is more penalty on larger coefficients than on smaller ones. However, it is possible to strategically construct a reweighted l₁-norm such that non-zero coefficients are penalized in a way that the cost further resembles the l₀-norm. By putting large weights on small entries, the solution concentrates on entries with small weights, non-zero entries are discouraged in the recovered signal, and a cost function that is more similar to an l₀-norm cost function can be solved (Candes et al., 2008). To find such weights for l₁-norm cost function, Candes et al. (2008) have proposed an iterative algorithm for enhancing the sparsity using reweighted l₁ minimization, which solves

$${min}_{u} {‖ u ‖}_{0} .$$

This algorithm is based on Fazel's “log-det heuristic” algorithm for minimizing the number of non-zero entries of a vector (Fazel, 2002) and the convergence of this log-det heuristic algorithm has been studied in Lobo et al. (2007). Hence, we use the algorithm by Candes et al. (2008) such that the constraints in the optimization problem in Equation (3) are satisfied:

1. Initialize the diagonal matrix W⁽⁰⁾ with entries w⁽⁰⁾ᵢ=1, i=1, . . . , n on the diagonal and zeros elsewhere

2. Solve

$${u}^{( l )} = arg { min}_{u} {‖ {W}^{( l )} u ‖}_{1}$$

subject to the constraints in Equation (3)

3. Update the weights

$${w}_{i}^{( l + 1 )} = \frac{1}{\vert {u}_{i}^{( l )} \vert + ε} , i = 1 , … , n$$

4. Iterate till l reaches a certain number of iterations. Otherwise, increment l and go to step 2.

The idea is, that by solving

$${u}^{( l + 1 )} = arg { min}_{u} {\sum }_{i = 1}^{n} \frac{\vert {u}_{i} \vert}{\vert {u}_{i}^{( l )} \vert + ε}$$

iteratively, the algorithm attempts to solve for a local minimum of a concave penalty function that is more similar to the l₀-norm (Candes et al., 2008). ε is used to ensure that weights on the recovered zero entries will not be set to ∞ at the next step, which would prevent us from obtaining estimates at the next step. ε should be slightly larger than the expected non-zero amplitudes of the signal that is to be recovered, and a value of at least 0.001 is recommended (Candes et al., 2008). This algorithm does not always find the global minimum and as ε→0, the likelihood of stagnating at an undesirable local minimum increases (Candes et al., 2008). For ε values closer to zero, the iterative reweighted l₁-norm algorithm stagnates at an undesirable local minimum (Candes et al., 2008).

The optimization problem in Equation (1) was analyzed further via four examples. The first example analyzes the case that the optimization formulation in Equation (1) is selecting the control such that the state (i.e., the blood cortisol concentration) is bounded between constant lower and upper bounds to illustrate the idea that the formulation in Equation (1) can achieve impulse control. Then, the case in which the upper and lower bounds have harmonic profiles with a circadian rhythm was studied. Using the iterative algorithm for enhancing the sparsity by reweighted l₁ minimization (Candes et al., 2008), the optimization problem in Equation (1) was solved over a time period T and the solution was updated after a time period τ/2. The process was repeated for a 24-hour period. λ, γ, ε, τ, and lower and upper bounds are given inTables 1-3. Since empirically the algorithm converges in 10 iterations for the formulation in this study, we use l=10 when running the algorithm. Numerical analysis was performed in MATLAB R2011b and using CVX (Grant and Boyd, 2008, 2014).

TABLE 1
Model parameters for examples of optimization problem (Equation 1)

| Example | λ(min⁻¹) | γ(min⁻¹) | $ε ( \frac{ug}{dL min} )$ | τ(min) |
|---|---|---|---|---|
| 1 | 0.0585 | 0.0122 | 0.01 | 360 |
| 2 | 0.0585 | 0.0122 | 0.0055 | 360 |
| 3 | 0.0585 | 0.0122 | 0.0075 | 360 |
| 4 | 0.1248 | 0.0061 | 0.0075 | 360 |

The parameters λ and γ are, respectively, the infusion rate of cortisol into the circulation from the adrenal glands and the clearance rate of cortisol by the liver, and were both obtained from Faghih et al. (2014). The parameter ε provides stability for the iterative algorithm for enhancing the sparsity by reweighted l₁ minimization (Candes et al., 2008), and τ is the period over which we solve the iterative algorithm.

+0 TABLE 2
Upper bounds for examples of optimization problem (Equation 1)

| Exam- ple | $q ( t ) ( {}^{\underline{u g}} dl )$ |
|---|---|
| 1 | 14 |
| 2 | $5.3782 + 0.3939 sin ( \frac{2 πt}{1440} ) - 3.5550 cos ( \frac{2 πt}{1440} ) - 0.5492 sin ( \frac{2 πt}{720} ) +$ |
| | $1.0148 cos ( \frac{2 πt}{720} )$ |
| 3 | $8.6051 + 3.0306 sin ( \frac{2 πt}{1440} ) - 5.0931 cos ( \frac{2 πt}{1440} ) - 1.8151 sin ( \frac{2 πt}{720} ) -$ |
| | $1.6570 cos ( \frac{2 πt}{720} )$ |
| 4 | $8.6051 + 3.0306 sin ( \frac{2 πt}{1440} ) - 5.0931 cos ( \frac{2 πt}{1440} ) - 1.8151 sin ( \frac{2 πt}{720} ) -$ |
| | $1.6570 cos ( \frac{2 πt}{720} )$ |

q(t) is the upper bound on the cortisol level.

TABLE 3
Lower bounds for examples of optimization problem (Equation 1)

| Ex- ample | $h ( t ) ( \frac{ug}{dl} )$ |
|---|---|
| 1 | 6 |
| 2 | $3.2478 - 0.7813 sin ( \frac{2 πt}{1440} ) - 2.8144 cos ( \frac{2 πt}{1440} ) - 0.2927 sin ( \frac{2 πt}{720} ) +$ |
| | $1.3063 cos ( \frac{2 πt}{720} )$ |
| 3 | $5.5065 + 1.5544 sin ( \frac{2 πt}{1440} ) - 4.3112 cos ( \frac{2 πt}{1440} ) - 1.6355 sin ( \frac{2 πt}{720} ) -$ |
| | $0.9565 cos ( \frac{2 πt}{720} )$ |
| 4 | $5.5065 + 1.5544 sin ( \frac{2 πt}{1440} ) - 4.3112 cos ( \frac{2 πt}{1440} ) - 1.6355 sin ( \frac{2 πt}{720} ) -$ |
| | $0.9565 cos ( \frac{2 πt}{720} )$ |

The methods and data are described in detail in the Examples. To illustrate that the methods described herein resulted in impulse control, we use constant lower and upper bounds and show that the proposed method achieves impulse control and a state that has a pulsatile profile. This example is not physiological and is used to help the reader better understand the type of results this type of approach generates. In the second example, we show a method that corresponds to a healthy subject and leads to impulse control. The secretory events and cortisol levels are in agreement with physiologically plausible profiles in healthy human data, and the obtained solution is optimal. Moreover, we illustrate another example that corresponds to a healthy subject and achieves impulse control. In this example, while the secretory events and cortisol levels are physiologically plausible, the obtained solution is optimal over the first 20 hours. Finally, we provide an example that illustrates a case in which the number of pulses is not within a physiologically plausible range (i.e., an abnormality) while impulse control is achieved.

### EXAMPLES

### Example 1

### Impulse Control via Equation 1

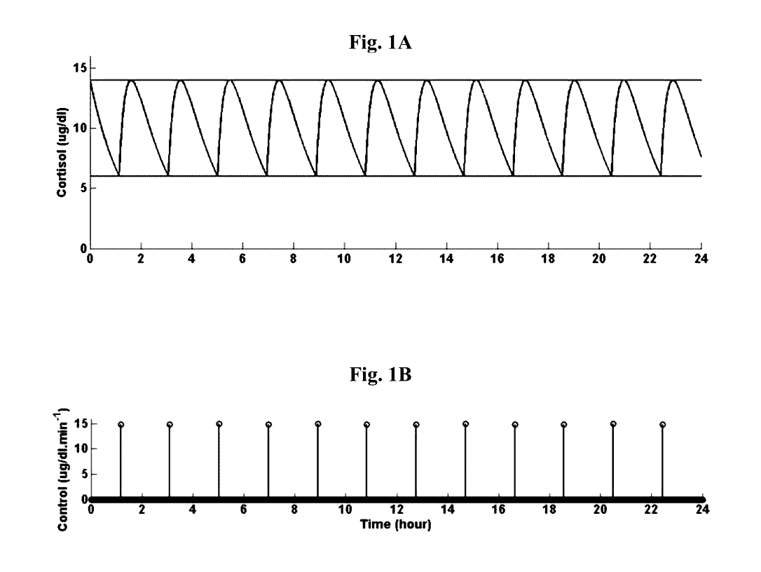

Assuming that the upper and lower bounds are constant, the optimal solution is achieved when the initial condition starts at the upper bound; then, when the state decays to the lower bound, an impulse causes a jump in the state which brings it back to the upper bound, and then again the state decays to the lower bound and the same jump to the upper bound again occurs, and the same process keeps repeating. FIG. 1 shows that solving the optimization problem (Equation 1) for constant upper and lower bounds using the parameters given for Table 1 and the upper and lower bounds provided in Tables 2 and 3, respectively, results in impulse control. There are 12 constant impulses obtained over a 24-h period, which occur periodically. This example illustrates that the optimization formulation in Equation (1) can achieve impulse control and pulsatile cortisol release using a low energy input.

### Example 2

### Impulse Control and Pulsatile Cortisol Release

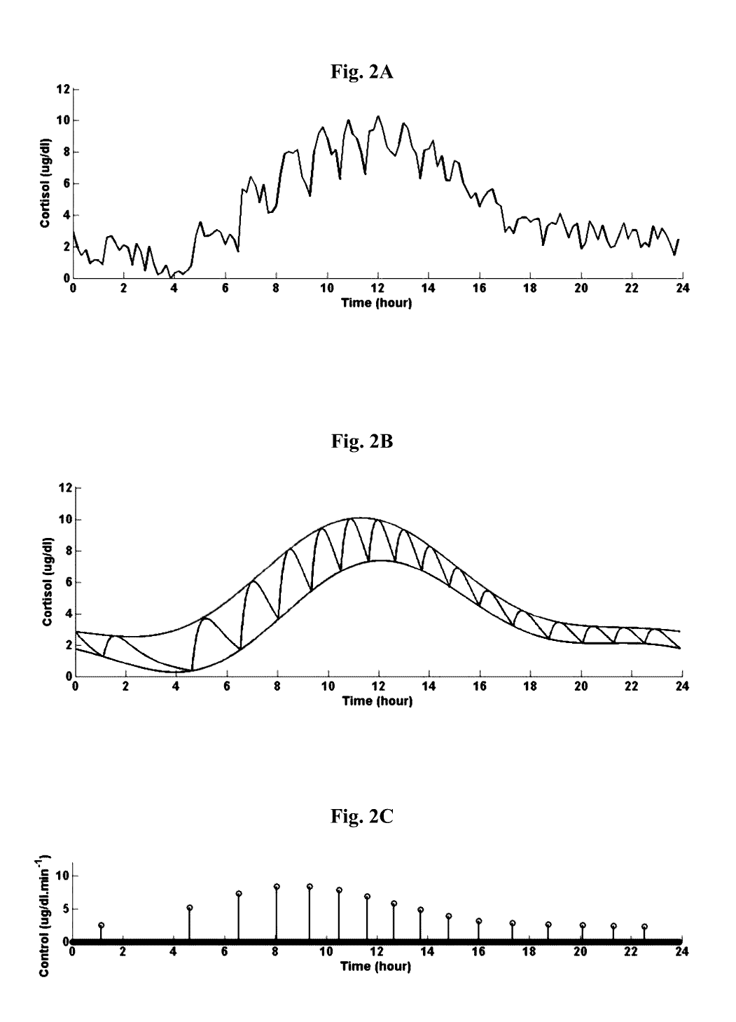

In healthy humans, cortisol levels have regular periodic time-varying patterns that consist of episodic release of secretory events with varying timings and amplitudes in a regular diurnal pattern. FIG. 2 shows that solving the optimization problem (Equation 1) for two-harmonic bounds with a circadian rhythm, using the parameters given for Example 2 in Table 1 and the upper and lower bounds provided in Tables 2 and 3, respectively, the obtained control is impulse control. FIG. 2 also displays that adding a zero mean Gaussian measurement error with a standard deviation of σ=0.45 to each simulated data point and recording the cortisol levels every 10 min (which is comparable to measurement noise and sampling interval of cortisol data in human subjects, Faghih et al., 2014), the obtained cortisol profile resembles cortisol human data provided in Faghih et al. (2014). There are 16 impulses over a 24-h period with time-varying circadian amplitudes and ultradian timings;

the obtained control is within the physiologically plausible range of 15-22 pulses (Veldhuis et al., 1989; Brown et al., 2001). The impulses are more frequent during the day and have higher amplitudes during the day than in night time. Obtained cortisol levels are low at night. Then, around 6 AM, cortisol levels increase, reaching higher values between 10 AM and 12 PM, followed by a gradual decrease throughout the day reaching low values at night. The obtained control and state are optimal; the state starts at the upper bound and decays to the lower bound at which point an impulse causes a jump in the system that results in increasing the state, and the state reaches the upper bound. Then, the state decays again to the time-varying lower bound and this process repeats. This example illustrates that the optimization formulation in Equation (1) can achieve impulse control and pulsatile cortisol release, using a low energy input, and generate secretory events and cortisol levels that have physiologically plausible profiles similar to those observed in healthy human data.

### Example 3

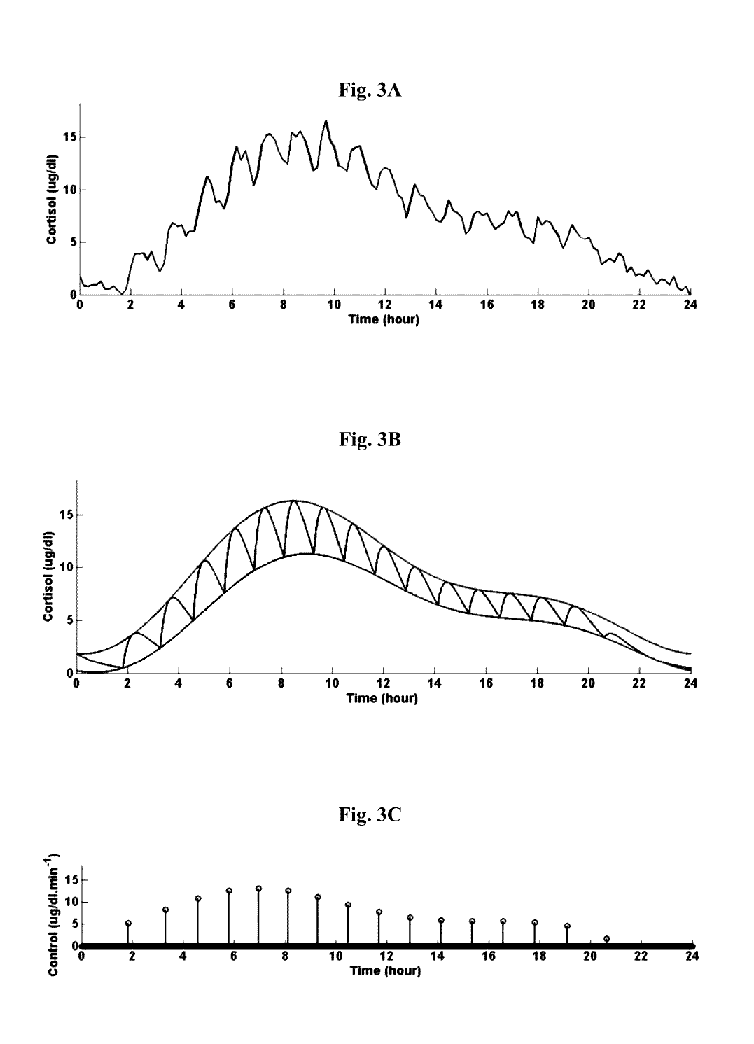

In this example, we consider different lower and upper bounds compared to Example 2 while keeping λ and γ to values used in Example 2. FIG. 3 shows that solving the optimization problem (Equation 1) for two-harmonic bounds with a circadian rhythm, using the parameters given for Example 3 in Table 1 and the upper and lower bounds provided in Tables 2 and 3, respectively, the obtained control is impulse control. FIG. 3 also displays that adding a zero mean Gaussian measurement error with a standard deviation of σ=0.45 to each simulated data point and recording the cortisol levels every 10 min (which is comparable to measurement noise and sampling interval of cortisol data in human subjects, Faghih et al., 2014), the obtained cortisol profile resembles cortisol human data provided in Faghih et al. (2014). Sixteen impulses are obtained over 24 h which is within the physiological range of 15-22; these impulses have time-varying circadian amplitudes and ultradian timings. The impulses have higher amplitudes and are more frequent between 4 AM and 12 PM. The obtained cortisol levels are low at night. Then, the cortisol levels increase, reaching higher values between 7 AM and 11 AM, followed by a gradual decrease throughout the day, reaching low values at night. This example illustrates that the optimization formulation in Equation (1) can achieve impulse control and pulsatile cortisol release using a low energy input, and generates secretory events and cortisol levels that have physiologically plausible profiles similar to those observed in healthy human data. The control and state obtained in the first 20 h are optimal. A low energy control is recovered that keeps the cortisol levels within the desired bounds.

### Example 4

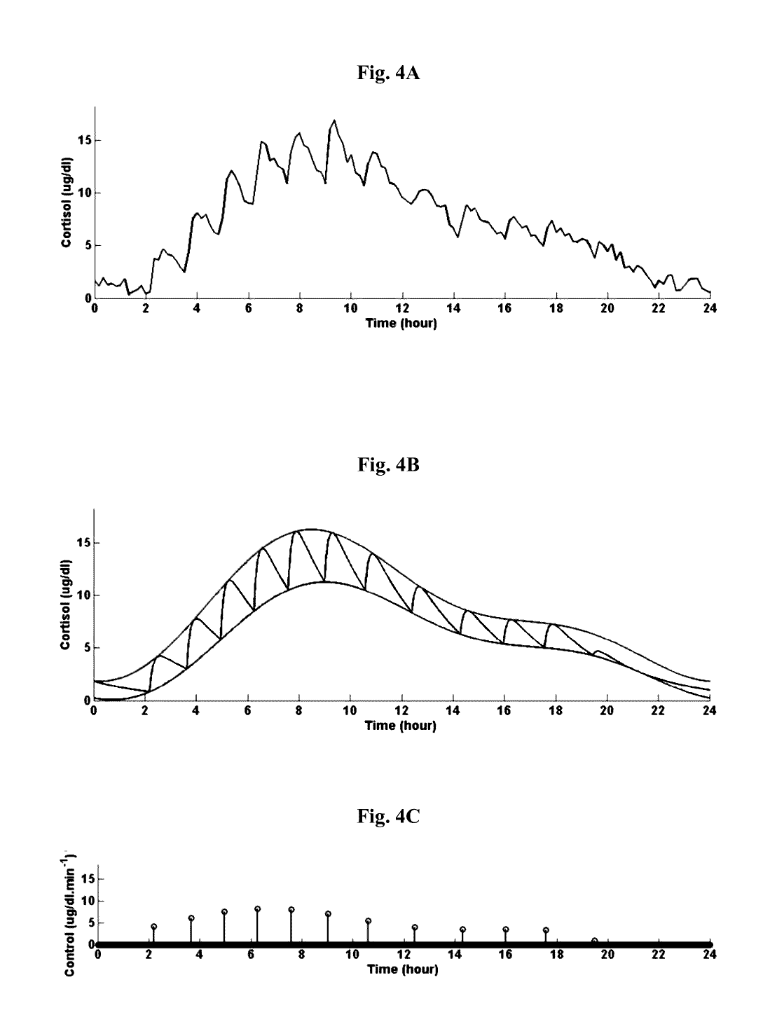

In this example, we keep the lower and upper bounds the same as the values we used in Example 3 while using values for λ and γ that result in higher infusion of cortisol and lower clearance of cortisol compared to Example 3. FIG. 4 shows that solving the optimization problem (Equation 1) using the parameters given for Example 4 in Table 1 and the upper and lower bounds provided in Tables 2 and 3, respectively, the obtained control is impulse control. FIG. 4 also displays that adding a zero mean Gaussian measurement error with a standard deviation of σ=0.45 to each simulated data point and recording the cortisol levels every 10 min (which is comparable to measurement noise and sampling interval of cortisol data in human subjects Faghih et al., 2014), the obtained cortisol profile resembles cortisol human data provided in Faghih et al. (2014). Twelve impulses are obtained over 24 h where the impulses have lower amplitudes and are less frequent compared to the impulses obtained in Example 3. The obtained impulses still have time-varying circadian amplitudes and ultradian timings. The number of pulses has decreased compared to Example 3 which was expected as cortisol is cleared faster in this example. While the number of these pulses are not within the physiological range reported for healthy subjects, the obtained cortisol levels are still within the desired range. Cortisol levels are low at night, then increase, reaching higher values between 6 AM and 10 AM, followed by a gradual decrease throughout the day, reaching low values at night. The peak values of cortisol levels change and on average in this example the cortisol levels have lower values, and this might illustrate a case of cortisol deficiency. Also, in this example, the optimization formulation in Equation (1) results in impulse control and pulsatile cortisol release using a low energy input. The control and state obtained in the first 19 h are optimal. A low energy control is recovered that keeps the cortisol levels within the desired bounds.

It is shown herein that a method of relaying information on cortisol released in pulses is an optimal approach as opposed to continuous signaling. In this work, we demonstrated this concept by proposing an optimization formulation for a physiologically plausible controller in the anterior pituitary that achieves impulse control as the optimal solution. In the proposed formulation, we assumed that there is a time-varying upper bound on the cortisol levels in the blood. Also, we assumed that the cortisol levels in the blood should be above a time-varying circadian threshold to achieve normal regulation of the HPA axis. We assumed that the lower bound and upper bound on the cortisol levels are two-harmonic functions with periods of 12 and 24 h that are controlled by the circadian rhythm. However, the upper bound and the lower bound for cortisol secretion could have multiple harmonics, and this assumption is only considering the most significant periods in cortisol release. Moreover, we considered the first-order dynamics underlying cortisol secretion. We have shown that the proposed optimization formulation yields impulse control as its optimal solution. The number, timing, and amplitude of the recovered secretory events in the proposed optimization problem are physiologically plausible. Moreover, the obtained cortisol profile is in agreement with the circadian rhythm observed in healthy human data. The iterative algorithm for enhancing the sparsity by reweighted l1 minimization (Candes et al., 2008) does not always find the global minimum and might stagnate at an undesirable local minimum; we employed this algorithm to solve examples of optimization problems formulated in Equation (1) to show that the formulation in Equation (1) achieves impulse control as observed in cortisol levels. However, the optimization problem in Equation (1) can be solved using other methods as well.

To validate this mathematical characterization using experiments, the parameters for a subject can be recovered to obtain lower and upper bounds on cortisol levels in a healthy subject. Next, using a pulse controller a cortisol profile that stays within the lower and upper bounds in a healthy subject may be obtained in a diseased subject.

While we proposed a simple optimization formulation that can achieve impulse control, it is possible to obtain impulse control using more complex formulations by either assuming that the system is a switched system with different rates or assuming that the nature of the system is impulsive and there is no continuous control. We assumed that the infusion and clearance rates are constant; however, the system can be a switched system with different infusion and clearance rates. Abrupt changes in the infusion and clearance rates could also result in impulse control. For example, if the infusion rate of cortisol from the adrenal glands starts from a constant level at wake and decreases abruptly to a new constant level, a very large level of cortisol should be produced in a short time so that the desired cortisol level can still be achieved. There could be multiple abrupt changes in the infusion rate throughout the day, and there might be an infusion rate reset to a high level at the beginning of sleep. Another example that could possibly result in impulse control is when the clearance starts at a constant level, and increases abruptly to a new constant level; then, a very large level of cortisol should be produced in a short time so that the desired cortisol level can still be achieved. There could be multiple such abrupt changes in the clearance rate throughout the day, and the clearance rate might be reset to a low level at the beginning of sleep. Another scenario could be that both the infusion and the clearance rates could be starting from a constant level and change abruptly to different levels periodically. In that case, the overall effect is that cortisol gets cleared faster or cortisol gets infused to the blood more slowly, and at such moments a very large cortisol level should be released for a short period of time to maintain the desired cortisol level. Such situations could possibly achieve impulse control as long as there is not an upper bound on the control variable; a mathematical example of a model with a time-varying rate that achieves impulse control is given in Sethi and Thompson (2000), and the maximum principle is used to find the optimality conditions for this problem. Moreover, it is possible that pulsatile inputs arise from the nature of the system, and the hormone system might be designed such that the input to the system can only be impulsive where the timing of the impulses are functions of the states and are not activated until a resetting condition is satisfied. A mathematical example of such a model is given in Wang and Balakrishnan (2008) where the cost function minimizes the energy in the input and the state, and calculus of variations is used to find the optimality conditions. Also, another possibility is that the body is solving a weighted l1 cost function where different costs are associated with the control at different times of the day (e.g., the weights obtained at convergence when using the reweighted algorithm).

In this study, for modeling cortisol secretion, we proposed a physiologically plausible optimization formulation for a controller in the anterior pituitary. The method is applicable to other endocrine hormones that are released in pulses. For example, the proposed optimization formulation can be tailored to include the constraints underlying thyroid hormone secretion or gonadal hormone secretion or growth hormone secretion. The transcriptional program stimulated by hormone pulses is very different from constant hormone treatment and some disorders are associated with hormone pulsatility. Hence, understanding the underlying nature of the pulsatile release of these hormones via mathematical formalization can be beneficial to understanding the pathological neuroendocrine states and treating some hormonal disorders.

In addition to contributing to the scientific advances in understanding cortisol regulation in daily rhythms, we provide methods and devices to devise pulsatile control interventions instead of continuous controllers for treating cortisol disorders. Traditional control-theoretic methods do not normally consider the intermittent control that is observed in pulsatile control of cortisol release. Instead of developing a controller that tracks the desired cortisol levels, we have described a formulation for a controller that maintains the cortisol levels within certain upper and lower bounds. Our study formalizes, mathematically, the pulsatile controller underlying cortisol secretion, and through a simulation study we show that our formulation can control the cortisol levels to remain within the desired bounds while having the circadian and the ultradian rhythms underlying cortisol secretion. The proposed formulation/device is an intermittent controller for curing cortisol deficiency. The proposed intermittent controller can be used to control the pathological problems related to cortisol by including the first-order kinetics of the medicine that will be injected to the patient to control cortisol levels, and then using compressed sensing algorithms to recover the secretory release of cortisol in the patient. In this case there will be two sets of pulses that control cortisol levels: (i) external pulses that are injected to the patient (ii) pulses that are secreted as a part of the natural control system underlying cortisol secretion.

A patient who suffers from Addison's disease takes cortisone once or twice a day for their cortisol deficiency. This dosing system is not optimal because it doesn't reflect physiological conditions. In contrast, an impulse controller of the invention can be used to control the cortisol levels optimally.

### REFERENCES

1. Brown E. N., Meehan P. M., Dempster A. P. (2001). A stochastic differential equation model of diurnal cortisol patterns. Am. J. Physiol. Endocrinol. Metab. 280, E450-E461. Available online at: ajpendo.physiology.org/content/280/3/E450.long
2. Candes E. J., Wakin M. B., Boyd S. P. (2008). Enhancing sparsity by reweighted l₁ minimization. J. Fourier Anal. Appl. 14, 877-905. 10.1007/s00041-008-9045-x
3. Faghih R. T. (2010). The Fitzhugh-Nagumo Model Dynamics with an Application to the Hypothalamic Pituitary Adrenal Axis. S.M. thesis, Massachusetts Institute of Technology, Cambridge.
4. Faghih R. T. (2014). System Identification of Cortisol Secretion: Characterizing Pulsatile Dynamics. Ph.D. thesis, Massachusetts Institute of Technology, Cambridge.
5. Faghih R. T., Dahleh M. A., Adler G. K., Klerman E. B., Brown E. N. (2014). Deconvolution of serum cortisol levels by using compressed sensing. PLoS ONE 9:e85204. 10.1371/journal.pone.0085204
6. Faghih R. T., Savla K., Dahleh M. A., Brown E. N. (2011). A feedback control model for cortisol secretion, in 2011 Annual International Conference of the IEEE Engineering in Medicine and Biology Society (Boston, Mass.:), 716-719.
7. Fazel M. (2002). Matrix Rank Minimization with Applications. Ph.D. thesis, Stanford, Calif.: Stanford University.
8. Grant M., Boyd S. (2008). Graph implementations for non-smooth convex programs, in Recent Advances in Learning and Control, Lecture Notes in Control and

Information Sciences, eds Blondel V., Boyd S., Kimura H., editors. (Springer-Verlag Limited;), 95-110. Available online at: stanford.edu/˜boyd/graph_dep.html
9. Grant M., Boyd S. (2014). CVX: Matlab Software for Disciplined Convex Programming, Version 2.1. Available online at: cvxr.com/cvx
10. Lightman S. L., Conway-Campbell B. L. (2010). The crucial role of pulsatile activity of the HPA axis for continuous dynamic equilibration. Nat. Rev. Neurosci. 11, 710-718. 10.1038/nrn2914
11. Lobo M. S., Fazel M., Boyd S. (2007). Portfolio optimization with linear and fixed transaction costs. Ann. Oper. Res. 152, 341-365. 10.1007/s10479-006-0145-1
12. McMaster A., Jangani M., Sommer P., Han N., Brass A., Beesley S., et al. (2011). Ultradian cortisol pulsatility encodes a distinct, biologically important signal. PLoS ONE 6:e15766. 10.1371/journal.pone.0015766
13. Sethi S. P., Thompson G. L. (2000). Optimal Control Theory: Applications to Management Science and Economics. Boston, Mass.: Kluwer Academic Publishers.
14. Spiga F., Waite E. J., Liu Y., Kershaw Y. M., Aguilera G., Lightman S. L. (2011). ACTH-dependent ultradian rhythm of corticosterone secretion. Endocrinology 152, 1448-1457. 10.1210/en.2010-1209
15. Stavreva D. A., Wiench M., John S., Conway-Campbell B. L., McKenna M. A., Pooley J. R., et al. (2009). Ultradian hormone stimulation induces glucocorticoid receptor-mediated pulses of gene transcription. Nat. Cell Biol. 11, 1093-1102. 10.1038/ncb1922
16. Veldhuis J. D. (2008). Pulsatile hormone secretion: mechanisms, significance and evaluation, in Ultradian Rhythms from Molecules to Mind, eds Lloyd D., Rossi E. L., editors. (Springer;), 229-248.
17. Veldhuis J. D., Iranmanesh A., Lizarralde G., Johnson M. L. (1989). Amplitude modulation of a burstlike mode of cortisol secretion subserves the circadian glucocorticoid rhythm. Am. J. Physiol. Endocrinol. Metab. 257, E6-E14.
18. Walker J. J., Spiga F., Waite E., Zhao Z., Kershaw Y., Terry J. R., et al. (2012). The origin of glucocorticoid hormone oscillations. PLoS Biol. 10:e1001341. 10.1371/journal.pbio.1001341
19. Walker J. J., Terry J. R., Lightman S. L. (2010a). Origin of ultradian pulsatility in the hypothalamic-pituitary-adrenal axis. Proc. R. Soc. Lond. B Biol. Sci. 277, 1627-1633. 10.1098/rspb.2009.2148
20. Walker J. J., Terry J. R., Tsaneva-Atanasova K., Armstrong S. P., McArdle C. A., Lightman S. L. (2010b). Encoding and decoding mechanisms of pulsatile hormone secretion. J. Neuroendocrinol. 22, 1226-1238. 10.1111/j.1365-2826.2010.02087.x
21. Wang X., Balakrishnan S. (2008). Optimal neuro-controller synthesis for variable-time impulse driven systems, in American Control Conference, 2008 (Seattle, Wash.: IEEE), 3817-3822.

The foregoing written specification is considered to be sufficient to enable one skilled in the art to practice the invention. The present invention is not limited in scope by the examples provided, since the examples are intended as illustrations of various aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A method for delivering cortisol to a subject having adrenal insufficiency comprising detecting a real-time level of circulating cortisol in blood of a subject having adrenal insufficiency, administering intermittent doses of cortisol to the subject based the level of circulating cortisol wherein the cortisol is within a lower circadian limit of cortisol and wherein the dose of cortisol administered to the subject adjusts the dose of cortisol to a range within an upper circadian limit of cortisol.

2. The method of claim 1, wherein the subject has Addison's disease.

3. The method of claim 1, wherein the intermittent doses are pulses.

4. The method of claim 1, wherein the dose of cortisol is at a maximum of the upper circadian limit of cortisol.

5. The method of claim 1, wherein the upper and lower circadian limits of cortisol are based on upper and lower circadian limits of cortisol in healthy humans.

6. A close loop cortisol infusion system comprising a sensor that measures cortisol levels in real time in a subject having adrenal insufficiency, a control algorithm that determines the amount of cortisol needed to keep cortisol levels within a healthy range, and a cortisol infusion device for delivering intermittent doses of cortisol to the patient in response to the calculation of the required cortisol.

7. The system of claim 6, wherein the intermittent doses of cortisol are within a range between a lower circadian limit of cortisol and an upper circadian limit of cortisol.

8. The system of claim 6, wherein the intermittent doses of cortisol are at a maximum upper circadian limit of cortisol.

9. The system of claim 6, wherein the amount of cortisol needed to keep cortisol levels within a healthy range is calculated based on an impulsive system.

10. The system of claim 6, wherein the amount of cortisol needed to keep cortisol levels within a healthy range is calculated based on a switched system.

11. The system of claim 10, wherein the switched system calculates different infusion rates.

12. The system of claim 6, wherein the switched system calculates different clearance rates.

13. The system of claim 6, wherein the amount of cortisol needed to keep cortisol levels within a healthy range is calculated based on a cortisol input amount.

14. The system of claim 13, wherein the cortisol input amount is calculated based on an amount of cortisol that is naturally produced by the body and an amount of cortisol that is delivered from the close loop cortisol infusion system.

15. A system for the delivery of an analyte to a subject, the system comprising: a pulsatile endocrine hormone sensor configured to provide a sensor pulsatile endocrine hormone measurement signal representative of sensed pulsatile endocrine hormone; an analyte delivery device configured to deliver intermittent doses of an analyte to a subject in response to control signals; and a controller programmed to receive the sensor pulsatile endocrine hormone measurement signal and to provide a delivery control signal to the delivery device as a function of the received sensor pulsatile endocrine hormone measurement signal.

16. The system of claim 15, wherein the control model is a range of circadian levels of pulsatile endocrine hormone in a healthy human.

17. The system of claim 16, wherein the range of circadian levels of pulsatile endocrine hormone has a lower limit and an upper limit and wherein the delivery control signal delivers a signal to provide a dose near the upper limit of the range.

18. The system of claim 15, wherein the analyte is cortisol

19-28. (canceled)

29. A kit comprising
a pulsatile endocrine hormone sensor configured to provide a sensor pulsatile endocrine hormone measurement signal representative of sensed pulsatile endocrine hormone;
an analyte delivery device configured to deliver intermittent doses of an analyte to a subject in response to control signals; and
a controller programmed to receive the sensor pulsatile endocrine hormone measurement signal and to provide a delivery control signal to the delivery device as a function of the received sensor pulsatile endocrine hormone measurement signal in accordance with a control model.

30. A device comprising a pulsatile endocrine hormone sensor configured to provide a sensor pulsatile endocrine hormone measurement signal representative of sensed pulsatile endocrine hormone.

31-33. (canceled)
